(19) United States
(12) Patent Application Publication
(10) Pub. No.: US 2009/0259508 A1
(43) Pub. Date: Oct. 15, 2009

(54) METHOD AND SYSTEMS FOR OPTIMIZING SCHEDULED SERVICES

(75) Inventors: Judi A. Grupp, Northbrook, IL (US); R. Thomas Brady, Libertyville, IL (US)

Correspondence Address: SONNENSCHEIN NATH & ROSENTHAL LLP, P.O. BOX 061080, WACKER DRIVE STATION, WILLIS TOWER, CHICAGO, IL, 60606-1080, US

(73) Assignee: ACTIVECARE NETWORK LLC, Bannockburn, IL (US)

(21) Appl. No.: 12/101,015

(22) Filed: Apr. 10, 2008

(51) Int. Cl. G06Q 10/00

(52) U.S. Cl. 705/8

(57) ABSTRACT

An automated system that improves utilization of resources and increases the effectiveness of prescribed procedures by insuring that required resources are available at the time of a scheduled service.

### FIELD OF THE INVENTION

The present invention generally relates to administering scheduled services, and more particularly to automated scheduling, delivery, and processing of services and resources to recipients.

### BACKGROUND OF THE INVENTION

The administration of services and resources needed during the services requires the synchronization of many parties. For example, administering a medical service and pharmaceuticals needed during the medical service requires coordinating pharmaceutical providers, payers, treatment providers, patients, and the like. Typically, this process involves the patient scheduling a patient procedure with the patient's doctor based on a calendar time available to both the patient and the doctor. If the patient is to receive treatment at a location other than the doctor's office, then personnel and resources at the treatment location must be coordinated as well. After the appointment is scheduled, the doctor or care provider makes frequent telephone calls to the patient to verify that the patient will be available at the scheduled time and makes frequent telephone calls to the drug provider and other resource providers to verify that other resources will be available as well. Thus, conventional approaches are labor intensive and lead to the inefficient utilization of expensive resources.

Further, conventional methods and systems have no ability to integrate or operationalize a payer's specific benefit authorization rules to ensure appropriate utilization of the administration of pharmaceuticals and patient care. This is conventionally a manual approach that frequently underperforms, resulting in lost time and inconsistencies in delivery of medical services, ultimately increasing the cost of the procedure and a reduction in the quality. Conventional approaches were not developed to handle the complex medical and pharmaceutical rules that manage the use of pharmaceuticals and services.

In the scope of medical services, conventional approaches further limit care and pharmaceutical delivery to limited locations, such as at the patient's doctor's office, and do not provide feedback to the physician or payers to inform them that the procedure was completed. Instead, physicians and payers assume that the procedure was completed as planned and provide little or no reporting on the procedure.

Conventional approaches also result in similar drawbacks with respect to other types of services, such as training services, transportation services, corrections, and the like.

### SUMMARY OF THE INVENTION

Methods, systems, and articles of manufacture (or “the system”) consistent with the present invention provide an automated mechanism for improved utilization of resources in sites designated for services and increase the effectiveness of prescribed procedures by insuring that required resources are available at the time of a scheduled service. For example, in the context of a medical service, the patient, treatment provider, pharmaceuticals, and other resources are automatically coordinated to be available for a scheduled medical service. In addition to ensuring that the required resources are available, methods, systems, and articles of manufacture consistent with the present invention manage the processes of procedure authorization and comply with the associated rules and billing processes. For example, in the context of a medical service, the medical service and pharmaceutical delivery are coordinated with the patient's treating physician and payer and conducted in accordance with benefit authorization rules and billing processes. Resources and requirements for executing a scheduled service are monitored to alert or confirm to users of the system as to the ability of a scheduled service to be performed. If a resource becomes unavailable or an authorization is not received, then relevant parties are notified and corrective action is taken.

Users of the system, such as service recipients, providers, and payers, access the system via a network, such as the Internet using a secure web browser. The system asynchronously monitors and evaluates the condition of selected events needed to complete a service. Once the events meet their individual requirements, the relevant parties are notified of the service event status. The system interfaces to authorization rules, such as benefit authorization rules, and adjudication systems that are used to manage the administration of resources, such as pharmaceuticals, and services. Further, the system also interfaces to resource providers, such as pharmaceutical distribution locations.

The system beneficially schedules the service at one of a plurality of available locations. Further, the system insures that the required resources are available at the right time and place for the service. Yet further, the system provides increased efficiency, accuracy, and timeliness of information compared to conventional approaches. The various parties involved in the service, from ordering to resource administration, are kept informed of the status of relevant resources required to perform the service. When there is a change in status of a resource, the other resources are automatically notified of the change. The other resources, in turn are automatically adjusted to be available to perform the service at a rescheduled time. This dependent resource monitoring keeps the resources synchronized with the service that needs to be provided.

Methods, systems, and articles of manufacture consistent with the present invention are useful in the delivery and administration of any type of service, such as but not limited to medical services, including infusions, injections, vaccination, other medical services or treatments, training, diagnostics, laboratory analyses, pandemic services, and the like; training services; transportation services; product delivery; corrections services; consulting services; and the like. For example, the system is useful in the delivery and administration of pharmaceuticals to people who have a chronic condition or are in need of education or training related to a specific condition. This includes, for example, receiving the prescription, adjudication of the prescription, shipment and receipt of the pharmaceutical, delivery of the procedure, and report of the actions to the interested parties.

In accordance with methods consistent with the present invention, a method in a data processing system having a program for coordinating a service is provided. The method comprises the steps of: receiving a request to coordinate a service; identifying at least one location of a plurality of locations at which the service can be performed and at least one time at which the at least one location is available to perform the service; selecting a selected location from the identified at least one location to perform the service; scheduling a time to perform the service at the selected location from the at least one time at which the selected location is available; notifying a recipient of the service, the selected location, and at least one resource provider of the scheduled time; after the scheduled time is scheduled and before the scheduled time, periodically determining whether the recipient of the service and the selected location are available for the service at the scheduled time; after the scheduled time is scheduled and before the scheduled time, periodically determining whether a resource that is used during the service has arrived at the selected location before a predetermined time; when it is determined that at least one of the recipient and the selected location are not available at the scheduled time, notifying the recipient, the selected location, and a provider of the resource that the scheduled time is rescheduled to an adjusted scheduled time; and when it is determined that the resource has not arrived by the predetermined time, notifying the recipient, the selected location, and the provider of the resource that the scheduled time is rescheduled to the adjusted scheduled time.

In accordance with articles of manufacture consistent with the present invention, a computer-readable medium containing instructions that cause a data processing system to perform a method for coordinating a service is provided. The method comprises the steps of: receiving a request to coordinate a service; identifying at least one location of a plurality of locations at which the service can be performed and at least one time at which the at least one location is available to perform the service; selecting a selected location from the identified at least one location to perform the service; scheduling a time to perform the service at the selected location from the at least one time at which the selected location is available; notifying a recipient of the service, the selected location, and at least one resource provider of the scheduled time; after the scheduled time is scheduled and before the scheduled time, periodically determining whether the recipient of the service and the selected location are available for the service at the scheduled time; after the scheduled time is scheduled and before the scheduled time, periodically determining whether a resource that is used during the service has arrived at the selected location before a predetermined time; when it is determined that at least one of the recipient and the selected location are not available at the scheduled time, notifying the recipient, the selected location, and a provider of the resource that the scheduled time is rescheduled to an adjusted scheduled time; and when it is determined that the resource has not arrived by the predetermined time, notifying the recipient, the selected location, and the provider of the resource that the scheduled time is rescheduled to the adjusted scheduled time.

In accordance with systems consistent with the present invention, a data processing system is provided. The data processing system comprises: a memory having a program for coordinating a service that receives a request to coordinate a service, identifies at least one location of a plurality of locations at which the medical service can be performed and at least one time at which the at least one location is available to perform the service, selects a selected location from the identified at least one location to perform the service, schedules a time to perform the service at the selected location from the at least one time at which the selected location is available, notifies a recipient of the service, the selected location, and at least one resource provider of the scheduled time, after the scheduled time is scheduled and before the scheduled time, periodically determines whether the recipient of the service and the selected location are available for the service at the scheduled time, after the scheduled time is scheduled and before the scheduled time, periodically determines whether a resource that is used during the service has arrived at the selected location before a predetermined time, when it is determined that at least one of the recipient and the selected location are not available at the scheduled time, notifies the recipient, the selected location, and a provider of the resource that the scheduled time is rescheduled to an adjusted scheduled time, and when it is determined that the resource has not arrived by the predetermined time, notifies the recipient, the selected location, and the provider of the resource that the scheduled time is rescheduled to the adjusted scheduled time; and a processing unit that runs the program.

Other systems, methods, features, and advantages of the invention will become apparent to one with skill in the art upon examination of the following figures and detailed description. It is intended that all such additional systems, methods, features, and advantages be included within this description, be within the scope of the invention, and be protected by the accompanying drawings.

### BRIEF DESCRIPTION OF THE DRAWINGS

The accompanying drawings, which are incorporated in and constitute a part of this specification, illustrate an implementation of the invention and, together with the description, serve to explain the advantages and principles of the invention. In the drawings,

FIG. 1 is a block diagram of a data processing system consistent with the present invention;

FIG. 2 is a block diagram of an administrator computer system consistent with the present invention.

FIG. 3 is a block diagram of another user's computer system consistent with the present invention.

FIG. 4 is a functional block diagram showing the administrator program functionally interacting with other components of the data processing system.

FIG. 5 is a flow diagram depicting illustrative steps performed by the resource optimizer.

FIG. 6 is a screen shot of an illustrative contract readiness display screen.

FIG. 7 is a screen shot of an illustrative operative readiness display screen.

FIG. 8 is a screen shot of an illustrative clinical readiness display screen.

FIG. 9 is a flow diagram depicting illustrative steps performed by the scheduler.

FIG. 10 is a functional diagram shown asynchronous communication between the event processing engine and other components.

### DETAILED DESCRIPTION

Reference will now be made in detail to an implementation consistent with the present invention as illustrated in the accompanying drawings. Wherever possible, the same reference numbers will be used throughout the drawings and the following description to refer to the same or like parts.

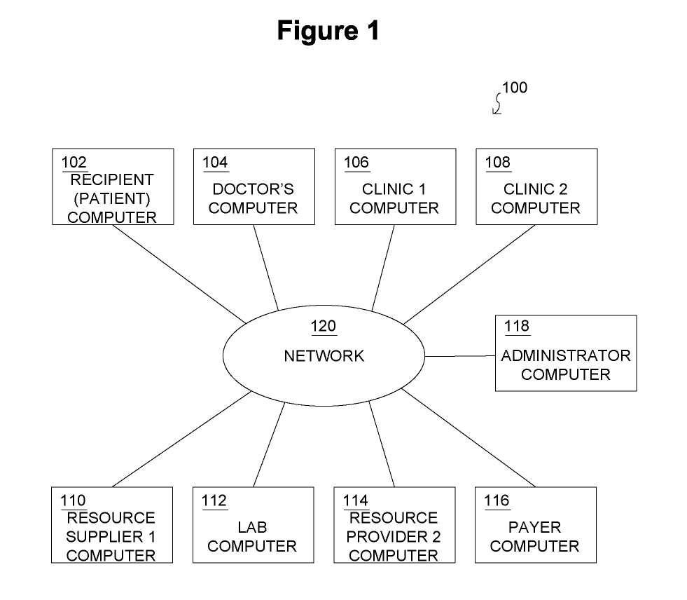

FIG. 1 is a block diagram of a data processing system 100 consistent with the present invention. Methods, systems, and articles of manufacture consistent with the present invention are described herein and in the drawings in an illustrative context of a patient receiving a medical service. This illustrative context is merely an illustrative example and methods, systems, and articles of manufacture consistent with the present invention are not limited thereto. Methods, systems, and articles of manufacture consistent with the present invention are useful in the delivery and administration of any type of service and resources, such as but not limited to medical services, including infusions, injections, other medical services or treatments, training, diagnostics, laboratory analyses, pandemic services, and the like; training services; transportation services; product delivery; corrections services; consulting services; and the like.

In the illustrative data processing system, a plurality of computer systems 102-118 communicate via a network 120, such as the Internet using secure web browsers. In an illustrative example, the data processing system includes a service recipient's computer 102 (e.g., a patient's computer), a patient doctor's computer 104, a clinic computer 106 at a first clinic, a clinic computer 108 at a second clinic, a drug supplier computer 110, a lab computer 112, an infusion provider computer 114, a payer computer 116, and an administrator computer 118. One having skill in the art will appreciate that the data processing system may include additional computers, such as additional patient and clinic computers, as well as computers located at other resources, such as at a drug manufacturer. Further, the scheduled service is not limited to a medical service. The respective computers may be located at alternative locations associated with the respective parties to the relevant service. For example, if the scheduled service is a training session for a group of teachers, the recipient computers may be located at the teachers' locations, the resource provider may be a text book distributor that provides text books instead of pharmaceuticals, and the training session may take place at a training center instead of a clinic. Further, the computers are not limited to being desktop or laptop devices. The computers and data processing systems may be other types of fixed or mobile computing devices, such as mobile telephones, personal data assistants, handheld personal computers, and the like.

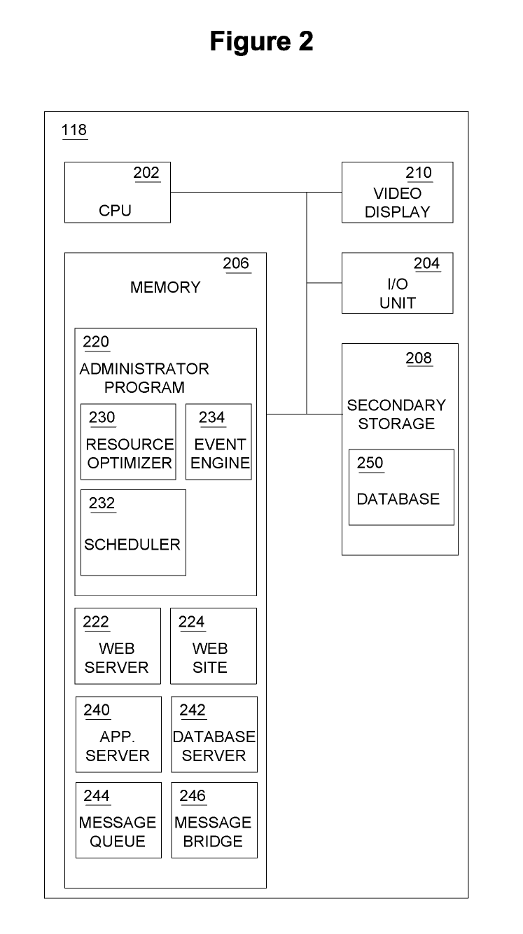

FIG. 2 shows a more detailed depiction of administrator computer 118. Administrator computer 118 comprises a central processing unit (CPU) 202, an input output I/O unit 204, a memory 206, a secondary storage device 208, and a video display 210. Administrator computer 118 may further comprise standard input devices such as a keyboard, a mouse or a speech processing means (each not illustrated).

The administrator computer's memory 206 includes an administrator program 220 for coordinating activities between the various resources associated with a medical service. Memory 206 also includes a web server program 222 that administers a web site 224, which may be accessed by the various computer systems 102-116. An application server program 240 receives messages via the web site and forwards the messages to administrator program using a message queue 244 and a message bridge 246. Each of these components will be described in more detail below.

In the illustrative example, the administrator program includes an event processing engine 234, which is the AptSoft Director™ software, which is manufactured by AptSoft Corporation of Burlington, Mass. In the illustrative example, the message queue 244 uses a Microsoft Messaging Queue protocol and the message bridge 246 is a Microsoft Messaging Queue Bridge. Further, in the illustrative example, the database server 242 is SQL Server 2005, which is manufactured by Microsoft of Redmond, Wash. Each of the product names described herein may be trademarks or registered trademarks of their respective owners. One having skill in the art will appreciate that the product names associated with the illustrative examples are merely illustrative. Alternative products may be used in a manner consistent with the present invention.

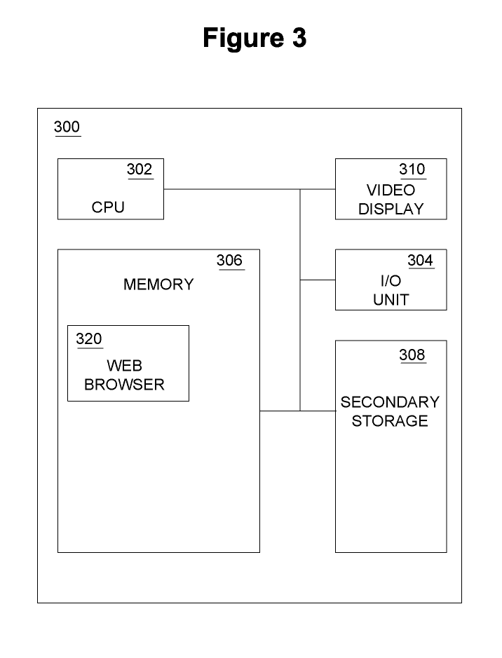

FIG. 3 shows a more detailed depiction of client computer 300 that is illustrative of computers 102-116 shown in FIG. 1. Client 300 comprises a central processing unit (CPU) 302, an input output I/O unit 304, a memory 306, a secondary storage device 308, and a video display 310. Client 300 may further comprise standard input devices such as a keyboard, a mouse or a speech processing means (each not illustrated). The client's memory 306 includes a web browser program 320 that may access the administrator's web site 224.

Each of the programs in the administrator computer's memory 206 and in the client's memory 306 will be described in more detail below. The programs may comprise or may be included in one or more code sections containing instructions for performing their respective operations. While the programs are described as being implemented as software, the present implementation may be implemented as a combination of hardware and software or hardware alone.

Although aspects of methods, systems, and articles of manufacture consistent with the present invention are depicted as being stored in memory, one having skill in the art will appreciate that these aspects may be stored on or read from other computer-readable storage media, such as secondary storage devices, like hard disks, floppy disks, CD-ROM, or other forms of ROM or RAM either currently known or later developed; or transmission media, such as a carrier wave received from a network such as the Internet. Further, although specific components of data processing system 100 have been described, one having skill in the art will appreciate that a data processing system suitable for use with methods, systems, and articles of manufacture consistent with the present invention may contain additional or different components.

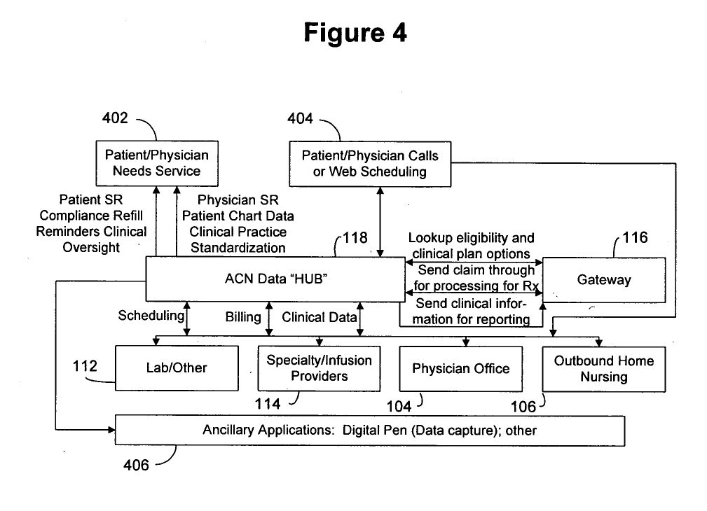

FIG. 4 is a functional block diagram that shows illustrative aspects consistent with the present invention of a patient receiving a drug infusion at one of the clinics. In the illustrative example, the selected clinic is a nursing home. As will be described in more detail below, when the patient needs to schedule an appointment for the drug infusion (item 402), the patient uses patient computer 102 to schedule the appointment at one of the clinics (item 404). Alternatively, the patient may receive the drug infusion at the patient's doctor's office, at an infusion provider, or at another location. In each case, administrator program on the administrator computer 118 coordinates the various relevant parties, which are also referred to as resources herein, through their respective computers so that the patient can receive the drug infusion at a scheduled time. The patient, using a web browser on the patient computer, accesses the web site that is administered by the administrator computer. The administrator's web site identifies dates and times during which one or more treatment locations are available to receive new appointments and presents appointment options to the patient. After receiving the patient's desired appointment time, the administrator program automatically notifies the selected treatment location, the patient's doctor, the payer (e.g., an insurer), the drug supplier via their respective computers. In the context of other types of services, the system similarly coordinates the scheduled service and delivery of resources with respective parties.

The administrator program can send reminders to the service recipient prior to the scheduled service, such as refill reminders to the client, as well as appointment reminders. Further, the administrator program forwards relevant information about the service recipient to other parties. For example, the administrator program may forward the patient's chart data to the patient's doctor. The administrator program also determines eligibility of the service recipient. For example, the administrator program may determine whether a patient is eligible for a medical treatment and identify clinical plan options for the patient, as well as confirm a valid prescription of an infused drug with the patient's doctor and payer. After the appointment is confirmed with the relevant parties, the administrator program coordinates delivery of any resources, such as drugs, supplies, medical examination, or lab tests from a supplier, to the service location, and notifies each relevant party after the service is completed. The administrator program also automatically manages claim processing and information, such as clinical information, for reporting. Further, the administrator program can interface with ancillary applications 406, such as data capture applications.

The administrator program includes a resource optimizer 232 component that manages scheduling of a service. As will be described in more detail below, the resource optimizer periodically reviews the condition of a set of events that need to occur in order to fulfill the appointment. For example, the resource optimizer periodically determines whether all relevant parties are available for the appointment at the scheduled time, and whether the resources, such as pharmaceuticals, will arrive on time.

The resource optimizer associates a service location, such as a clinic, with a status state. In the illustrative example, the service location may have a state of “potential”, “no interest”, “pending”, “contracted”, “ready”, “implemented”, or “suspended”. The service location may have fewer, greater, or alternative state than these illustrative states. Provider states are described below in the context of the illustrative example of a patient receiving a medical service at a client. However, this is merely an illustrative example and similar functionality consistent with the present invention may be used in connection with other types of services and providers.

In the illustrative example, a service location is ready to take appointments when it is in the “implemented” state. A single service location can have several different statuses assigned to it depending on the services being referenced. For example, Clinic A can have a “ready” state for vaccines, but a “pending” state for infusions. In the illustrative example, the treatment location can have one or more of the following illustrative states:
“Potential”—the provider has been identified (e.g., name/address/phone)
“No Interest”—a “potential” clinic that never reached a “pending” state. Clinic lost interest in the program.
“Pending”—in discussion but no contract document signed
“Contracted”—contract signed (e.g., per service)
“Ready”—All requirements (operation and clinical) have been met and the clinic is placed in “review” cue for an administrator to approve and make “active”
“Implemented”—available for scheduler to book appointments
“Suspended”—a clinic has been pulled from “ready” state. A reason why and who placed a clinic in this state is provided. A clinic can be set to “suspended” state either prior to an initial prod

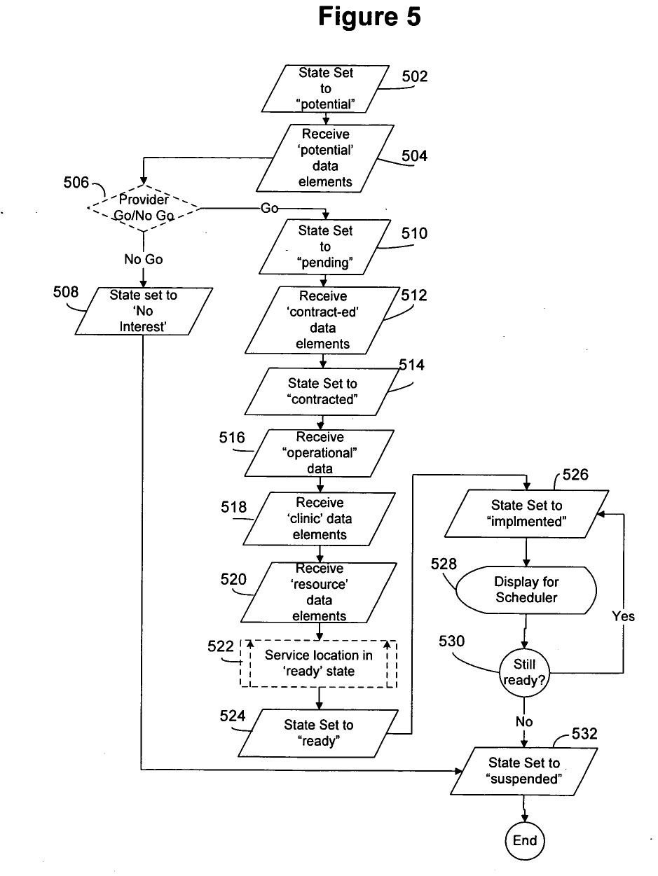

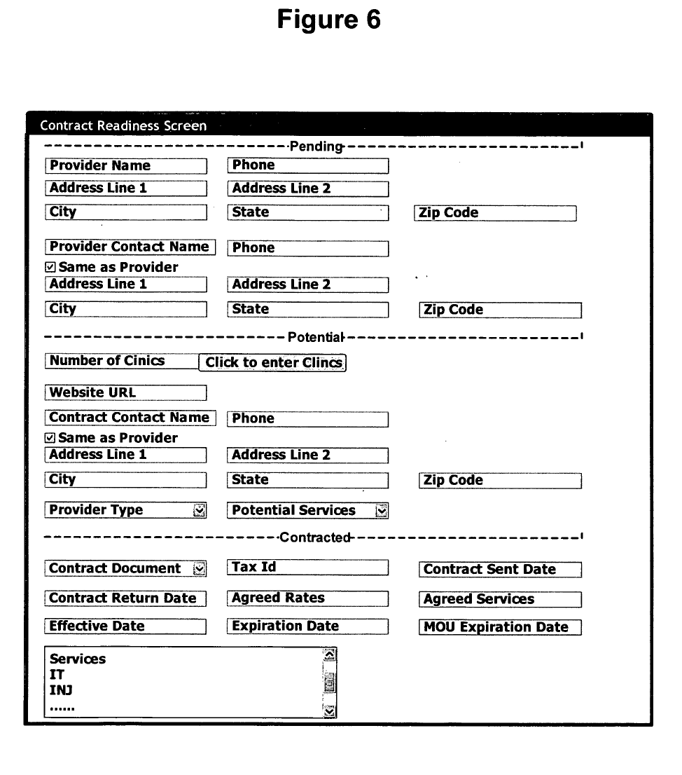

For a service location to be placed in the “implemented” state, a number of operational and clinical requirements must be satisfied. FIG. 5 is a flow diagram that depicts the illustrative steps performed by the resource optimizer for placing a service location in the “implemented” state. Process steps performed by the resource optimizer program are shown in solid lines, and process steps performed by a user are shown in phantom lines. Initially, a service location's state is set to “potential” (step 502). In this state, a service location has been identified as a potential service center, but has not yet entered into discussions to become a contractor to provide the service. A user enters information about the potential service provider into the administration computer (step 504). This information may include, for example, provider name, address, phone, contact information, and the like. FIG. 6 depicts an illustrative screen shot of a display screen presented by the resource optimizer program for receiving information about a medical service provider.

If the potential provider decides that the provider is not interested in contracting to provide services (step 506), then the resource optimizer program sets the provider's state to “no interest” (step 508). In this case, the provider will not appear as a potential provider of the service. However, if the provider is interested in moving forward with contract negotiations, then the provider's status is set to “pending” (step 510).

When the provider is in the “pending” state, the user gathers additional information about the provider and enters this information into the system using the resource optimizer program. In the illustrative example, these data elements may include, for example, the provider's clinic locations, web site address, contract contact information, provider type (e.g., ambulatory treatment center, urgent care center, convenient care center, dialysis center, home setting, physician's office, dialysis center, home setting, pharmacy, and the like), and potential services. In the illustrative example, potential services may include, for example, injection training (adult/pediatric), injections (adult/pediatric), infusion (adult/pediatric), vaccinations, infusion training, and the like.

After the contract has been negotiated and executed, a user submits information about the contract using the resource optimizer program (step 512). This contract-related information may include, for example, information about or a copy of the contract document (e.g., Memorandum of Understanding, Master Provider Agreement, Master Vaccine Agreement, and the like), the provider's tax identification number, agreed rates, agreed services, contract sent date, return date, expiration date, effective date, services (e.g., injection training, injections, infusions, vaccines, and the like), and Memorandum of Understanding expiration date. The resource optimizer also sets the provider status to “contracted” (step 514).

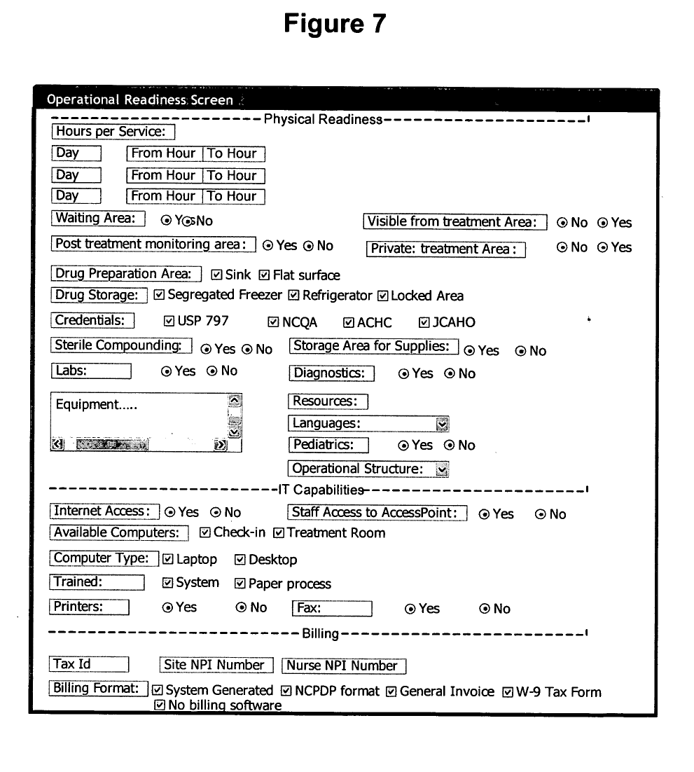

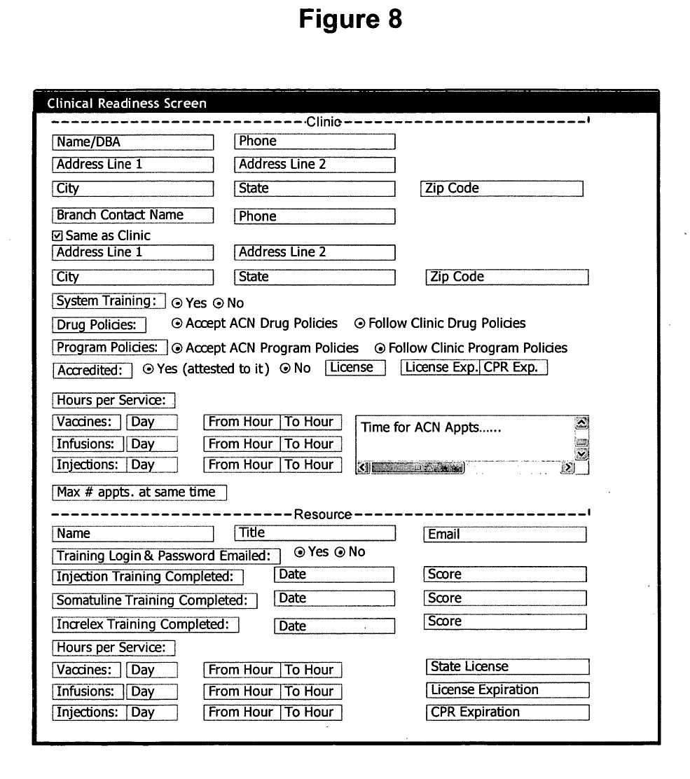

A contracted provider's facility must be in a “ready” state to be available to accept appointments. The resource optimizer program verifies a plurality of operational and clinical readiness criteria to verify that the provider's facility is ready. A user submits this operational and clinical readiness information to the resource optimizer. In the illustrative example, the resource optimizer program presents a number of display screens, such as those depicted in the screen shots shown in FIGS. 7 and 8, to receive the provider information. In the illustrative example, the resource optimizer receives operational readiness information, including physical readiness, information technology capabilities, and reimbursement and billing capabilities, as shown in FIG. 7; and clinical readiness information, including clinic information and resource information, as shown in FIG. 8. In the context of other types of services, the display screens may present alternative information relevant to the service requested.

In the illustrative example, the user enters the operational readiness information using the display screen shown in FIG. 7 (step 516). In the illustrative example, physical readiness information includes information on hours of operation; the waiting area, including whether the waiting are is visible from the treatment area; the post treatment monitoring area; whether there is a private treatment area; the drug preparation area, including whether there is a sink and a flat smooth surface; drug storage, including segregated freezers having logbooks, a refrigerator having a logbook, and whether drugs are kept in a locked area; credentials, such as USP 797, NCQA, ACHC, and JCAHO accreditations; sterile compounding; equipment types; supply storage areas; laboratories; resources, such as languages, pediatrics, operational structure; diagnostics; and the like.

In the illustrative example, the information technology component of the operational readiness information includes information on internet access; staff access to the scheduling software; computer availability, for example, at check-in, in treatment rooms, and available languages; computer types, such as laptop or desktop; whether personnel are trained on using the system and on paper processes if computers are not available; printers; fax machines; and the like.

The reimbursement/billing information component of the operational readiness information includes information on tax identification number; national provider identifier by site and by nurse; billing format, such as whether it is system generated, a National Council of Prescription Drug Programs (NCPDP) format, or a general invoice, whether there is billing software, a W-9 tax form; and the like.

In addition to gathering operation readiness information, the resource optimizer may collect clinical readiness information. In the illustrative example, this includes information about the treatment locations (step 518) and their available resources (step 520). The treatment location (e.g., clinic) information includes, for example, the name; address; branch contact; system training; policies, such whether it accepts and follows drug policies and whether it accepts and follows program policies; accreditation, including proof of accreditation, licenses, license expiration, and CPR expiration; contact phone; hours per service; time for appointments; maximum number of appointments at the same time; and the like. The available resources information includes, for example, name; title, such as registered nurse; email address; whether a training username and password have been sent; whether injection training has been completed, including date and score; whether specific drug training has been completed, including date and score; available work hours per service; available work days; state license; license expiration date; CPR expiration date; and the like.

When the operational and clinical readiness criteria satisfy predetermined requirements that are known to the resource optimizer (e.g., by comparing the received information to requirements in the database or a lookup table), then the provider status can be changed to “ready” (step 522). Accordingly, the resource optimizer changes the provider status to “ready” (step 524). An administrator confirms the provider's readiness and enters confirmation of readiness to the resource optimizers. When a provider's “ready” state is confirmed, the resource optimizer changes the provider state to “implemented” (step 526). After a provider is in the “implemented” state, it can receive service appointments through the scheduler. Accordingly, when a service recipient attempts to schedule an appointment for a particular service, if a provider location is “implemented” and meets the criteria for providing the treatment, then the provider location will be displayed to the recipient as an available service location (step 528). If the service location fails to meet one or more of the operational or clinical readiness criteria, then the service location status is changed to “suspended”. When the service location is in the “suspended” state, a recipient cannot schedule an appointment at that location.

To ensure that appointments can be met by providers, the resource optimizer monitors a set of conditions and reports on changes. These changes in conditions are referred to as events for purposes of this disclosure. If an event takes place, the resource optimizer notifies the relevant parties so that appropriate changes may be made, such as rescheduling an appointment. An event may be manually reported to the resource optimizer by a user of the system or automatically generated by the resource optimizer based on predefined business rules.

The resource optimizer applies a plurality of rule sets to detect events. In the illustrative example, the rule sets are defined in lookup tables that the resource optimizer analyzes upon a change in provider-related conditions. Illustrative rule sets are described below. One having skill in the art will appreciate that alternative or additional rules may be applied.

In the illustrative example, the resource optimizer detects change in provider status by applying the provider status rule set shown in Table 1.

TABLE 1
Provider Status Rule Set

| Parameters Met | Provider Status |
|---|---|
| provider name, address & | Potential |
| phone | |
| no interest past initial contact | No Interest |
| clinic locations, website, | Pending . . . notify |
| contract contact, provider | operational admin |
| type, potential services | |
| contract document, tax id, | Contracted . . . notify |
| agreed rates, agreed services, | clinical admin |
| contract info | |
| Operational & Clinical | Ready |
| requirements complete | |
| Ready clinics approved | Implemented |
| Ready clinic not approved to | Suspended |
| implemented or implemented | |
| clinic gets pulled from | |
| scheduler | |

As described above, a user enters information relating to the provider at each step in the process from the initial contact with the provider until the provider is “ready.” Referring to Table 1, when the identified parameters are met, the resource identifier changes the provider status accordingly. For example, the resource optimizer determines that a provider's status is “potential” after the provider name, address, and phone have been submitted.

The resource optimizer detects changes in provider contract document status by applying the contract document rule set shown in Table 2 and notifies relevant parties as described in the table. In the tables, “RO” is an acronym for resource optimizer, and “IT” is an acronym for information technology department. In the illustrative contract document rule set, for example, the resource optimizer identifies when the provider's memorandum of understanding will expire in 30 days and 60 days. When this condition is met, the resource optimizer notifies the contract administrator and the system administrator by, for example, displaying a web site alert or sending an e-mail to the party's e-mail address. Further, the resource optimizer notifies the contract administrator to check the contract status, with instructions to take a first predetermined action if the contract is signed (e.g., notify the resource optimizer that the contract has been signed) or a second predetermined action if the contract has not been signed (e.g., get the contract signed).

TABLE 2
Contract Document Rule Set

| | | Notify | Notify | |
|---|---|---|---|---|
| Parameter | Condition | (instructional) | (informational) | Action/Escalation |
| Memorandum of | > today - 30 | Contract | Admin | 1 - Site alert in RO (Contract |
| Understanding to | > today - 60 | | | Admin & Admin) |
| Expire | | | | 2 - Contract admin - check |
| (as defined under | | | | contract status |
| condition) | | | | 3 - If contract signed, . . . |
| | | | | 4 - If no contract, . . . |
| Memorandum of | > today's | Contract | Admin | 1 - Site alert in RO (Contract |
| Understanding | date | | | Admin & Admin) |
| Expiration Date | | | | 2 - Contract Admin - check |
| (alert - | | | | contract status |
| Memorandum of | | | | 3 - If contract signed, . . . |
| Understanding | | | | 4 - If no contract, . . . |
| Expired!!!) | | | | |
| Contract Renewal | > today - 90 | Contract | Admin | 1 - Site alert in RO (Contract |
| (as defined under | | | | Admin & Admin) |
| condition) | | | | 2 - Contract Admin - contact |
| | | | | Provider/Clinic . . . |
| Current Service(s) | new | Contract, | Admin, | 1 - Site alert in RO of New |
| provider/clinic to | service(s) | Operations, | information | Service(s) |
| add new service | | Clinical | technology | 2 - Contract admin contact |
| (infusions, | | | | Provider/Clinic |
| injections, etc.) | | | | 3 - If provider/clinic approves |
| | | | | new service, notify Contract |
| | | | | Admin & Clinical Admin. |
| | | | | Contract Admin - update contract |
| | | | | & Clinical Admin - start training |
| | | | | process. |
| | | | | 4 - If provider/clinic does not |
| | | | | approve new service, no action |

The resource optimizer detects change in provider operational readiness by applying the operational readiness rule set shown in Table 3 and notifies relevant parties as described in the table.

TABLE 3
Operational Readiness

| | | Notify | Notify | |
|---|---|---|---|---|
| Parameter | Condition | (instructional) | (informational) | Action/Escalation |
| Operational | per | Clinical | Admin, | 1 - Site alert in RO (Admin, Ops) |
| Readiness | drug/clinic | | information | 2 - Notify Clinical Admins |
| alert when all | | | technology | |
| attributes (on | | | | |
| matrix) are | | | | |
| complete. | | | | |
| Current drug list | new | Operations, | Admin, | 1 - Site alert in RO (Admin, Ops, |
| (per clinic) | drug/service | Clinical, | information | Clinical, Contract) |
| alert when new | added | Contract | technology | 2 - Notify Contract Admins - |
| drug/service added | | | | update contract to include new |
| to clinic | | | | drug/service |
| | | | | 3 - Ops & Clinical Admins |
| | | | | notified by Contract Admin |
| | | | | 4 - Ops & Clinical admins: get |
| | | | | Clinic ‘ready’ for new drug |

The resource optimizer detects change in provider clinical readiness by applying the clinical readiness rule set shown in Table 4 and notifies relevant parties as described in the table.

TABLE 4
Clinical Readiness

| | | Notify | Notify | |
|---|---|---|---|---|
| Parameter | Condition | (instructional) | (informational) | Action/Escalation |
| Clinical Readiness | per location | Clinical | Admin, | 1 - Site alert in RO (Admin, |
| alert when all | per | | information | Clinical) |
| attributes for | drug/service | | technology | 2 - Admin: approve ‘ready’ clinic |
| clinical readiness | | | | into ‘implemented’ state |
| (on matrix) | | | | 3 - IT: verify clinic available in |
| complete | | | | for Scheduler |
| Training - | > today - 30 | Clinical | Admin, | 1 - Site alert in RO (Admin, |
| Complete (Y/N) | > today - 60 | | information | Clinical) |
| to include all | > today - 90 | | technology | 2 - Clinical Admin: verify drug |
| training (drug, | | | | training complete . . . if not, get |
| system, ongoing, | | | | training complete |
| etc.) | | | | |
| Clinic Hours | Change in | Clinical, | Admin | 1 - Site alert in RO (Admin, |
| alert when change | hrs | information | | Clinical) |
| in hours (ex. open | | technology | | 2 - Clinical Admin: verify updated |
| later, closed | | | | clinic hours in RO (if not already |
| Fridays, etc.) | | | | done so by clinic) |
| | | | | 3 - Clinical Admin: check for |
| | | | | current appointments scheduled at |
| | | | | clinic . . . contact clinic/patient if |
| | | | | any overlaps to new hours |
| | | | | 4 - IT: verify new hours reflected |
| | | | | in Scheduler |
| Nurse Hours | Change in | Clinical, | Admin | 1 - Site alert in RO (Admin, |
| alert when change | hrs | information | | Clinical) |
| in hours (ex. no | | technology | | 2 - Clinical Admin: verify updated |
| longer working | | | | nurse hours in RO (if not already |
| Mondays, etc.) | | | | done so by clinic) |
| | | | | 3 - Clinical Admin: check for |
| | | | | current appointment scheduled at |
| | | | | clinic with specific nurse |
| | | | | services . . . contact clinic/patient if |
| | | | | any overlaps to new hours |
| | | | | 4 - IT: verify new hours reflected |
| | | | | in Scheduler |
| License expiration | > today - 30 | Clinical | Admin, | 1 - Site alert in RO (Admin, |
| (clinic/nurse) | > today - 60 | | information | Clinical) |
| alert when licenses | > today - 90 | | technology | 2 - Clinical Admin: verify |
| are close to | | | | clinic/nurse license expiration. |
| expiration date (as | | | | 3 - Clinical Admin: notify |
| defined under | | | | clinic/nurse of expiration and have |
| condition) | | | | them update license status. |

The resource optimizer detects change in clinic status by applying the clinic status rule set shown in Table 5 and notifies relevant parties as described in the table.

TABLE 5
Clinic Status

| | Provider | Notify | Notify | |
|---|---|---|---|---|
| Parameters Met | Status | (instructional) | (informational) | Action/Escalation |
| Operational/Clinical | Ready | Approver | Information | 1 - Site alert in RO (Admin, |
| Readiness | | | technology, | Operations, Clinical) |
| alert when all | | | Operations, | 2 - Admin reviews ‘ready’ clinics |
| attributes (per | | | Clinical | 3 - Clinics get approved to |
| drug/service) are | | | | ‘implemented’ or stay at ‘ready’ |
| complete | | | | |
| Operational/Clinical | Implemented | Information | Operations, | 1 - Site alert in RO (Admin, |
| Readiness | | technology | Clinical | Operations, Clinical) |
| Approved | | | | 2 - Admin approves ‘ready’ clinics |
| alert when clinics | | | | to ‘implemented’ |
| approved to | | | | 3 - Implemented clinics available |
| ‘implemented’ state | | | | to Scheduler |
| (per drug/service) | | | | |
| Operational or | Suspended | Operations, | Information | 1 - Site alert in RO (Admin, |
| Clinical Attribute | | Clinical, | technology | Operations, Clinical) |
| Change | | Admin | | 2 - Operations and Clinical |
| alert when clinic | | | | admins review attribute change |
| ‘suspended’ per | | | | 3 - If appointments |
| drug/service | | | | affected . . . clinics/patients |
| | | | | contacted. |
| | | | | 4 - suspends clinic if needed |
| Drug Change | Suspended | Clinical, | Information | 1 - Site alert in RO (Admin, |
| (ex. Drug gets | | Admin | technology | Operations, Clinical) |
| recalled, service hrs | | | | 2 - Clinical admins review drug |
| change for drug, | | | | change |
| etc.) | | | | 3 - If appointments |
| | | | | affected . . . clinics/patients |
| | | | | contacted. |
| | | | | 4 - suspends clinic if needed |

To schedule an appointment with one of the implemented providers, a user accesses the administrator program web site using their respective computer's web browser. As described above, appointments may be scheduled for a variety of services, such as but not limited to medical services, including infusions, injections, other medical services or treatments, training, diagnostics, laboratory analyses, pandemic services, and the like; training services; transportation services; product delivery; corrections services; consulting services; and the like. Alternatively, the user's appointment may be scheduled by another party, such as by a patient's doctor, a patient, a provider, or payer using their respective computer. The administrator program includes a scheduler 232 component that handles appointment scheduling tasks. Particular types of services or recipients may be restricted to certain providers. For example, a group of providers that are part of a particular medical network may receive patients, while other providers do not. As will be described below, the scheduler may filter providers based on user-inputted or other predetermined criteria.

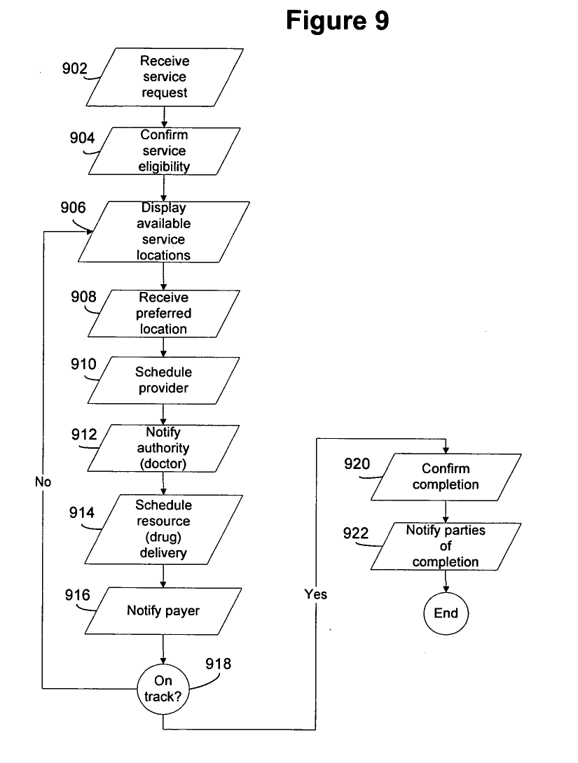

FIG. 9 is a flow diagram that depicts illustrative steps performed by the scheduler for scheduling an appointment for a service. First, the scheduler receives a request to schedule a service (step 902). The service request is received, for example, from a patient using the patient's computer's web browser to access the administrator web site. In the illustrative example, the request includes the intended recipient of the service, their payer group number, and may include additional information, such the name of their primary care giver. The scheduler then confirms that the service recipient is eligible for the requested service (step 904). This step is performed, for example, by sending requests via the network to the service recipient's doctor and payer to confirm that the recipient is eligible for the service. If the doctor and the payer return positive confirmations that the recipient is eligible, then the process continues to step 906. Otherwise, the recipient is notified that the service has been denied and the scheduling process terminates.

If the recipient is eligible for the service, then the scheduler identifies available providers who can provide the requested service (step 906). This is done, for example, using a lookup table to identify providers who provide the requested type of service and who have an “implemented” state. The scheduler filters providers based on, for example, drugs, drug service, drug or clinic parameters, clinic name, location, clinic state, and the like. The scheduler can further filter the available providers based on filter criteria selected by the user, such as location, resources available, and the like, or based on predetermined filter criteria, such as whether a patient is eligible to schedule with a particular provider network. The available providers and their respective open appointment times are displayed to the user, who selects a desired appointment time. Then, the scheduler receives the user selected provider and appointment time (step 908).

After receiving the desired appointment time and location, the scheduler notifies the relevant parties, such as the provider (step 910), the recipient's doctor (step 912), the drug provider (step 914), the recipient's payer (step 916), and the like. Additional parties may also be notified as may be required.

From the time at which the appointment was scheduled until the time at which the appointment takes place, the scheduler periodically requests confirmation from the relevant parties that the appointment will take place. For example, the scheduler may request confirmation from the patient that the patient will be able to attend the appointment, request confirmation from the provider that the location is still available, and request confirmation from the drug provider that the pharmaceuticals will be delivered on time. This can be done, for example, by sending reminders via e-mail or as postings on the administrator web site.

To confirm delivery of resources that need to arrive at the appointment location prior to the scheduled appointment, the scheduler identifies whether events have occurred that indicate that the resources have been delivered. For example, if a drug or other supplies, such as lab charts, medical exams, and the like, need to arrive at the treatment location prior to an appointment, the scheduler checks for a “drug received” event initiated by the treatment location. The scheduler and resource optimizer can receive event notifications in a variety of ways. For example, event notification can be received via user input to the administrator web site or via manual entry at the administrator computer.

The administrator program may send global messages or targeted messages to intended recipients. For example, the administrator program may send targeted messages to specific recipients based on specific conditions. In an illustrative example, the administrator program may alert various parties on a filtered or global basis regarding, for example, alerts on a drug recall other types of alerts, and identify the patients in each service location affected. In another illustrative example, the administrator program may push client lists and instructions to the relevant service locations in real time for supporting a particular program, such as a vaccine administration program.

When the administrator program sends a request for information to another computer on the system, it is unknown how long it will take a user of the other computer to respond with the requested information. For example, if the scheduler sends a message to a clinic's computer to determine whether a drug has arrived prior to an appointment, it may take some time for a nurse at the clinic to see the request for information and respond. To facilitate event processing, in the illustrative embodiment, the administrator program receives incoming messages from the web site asynchronously.

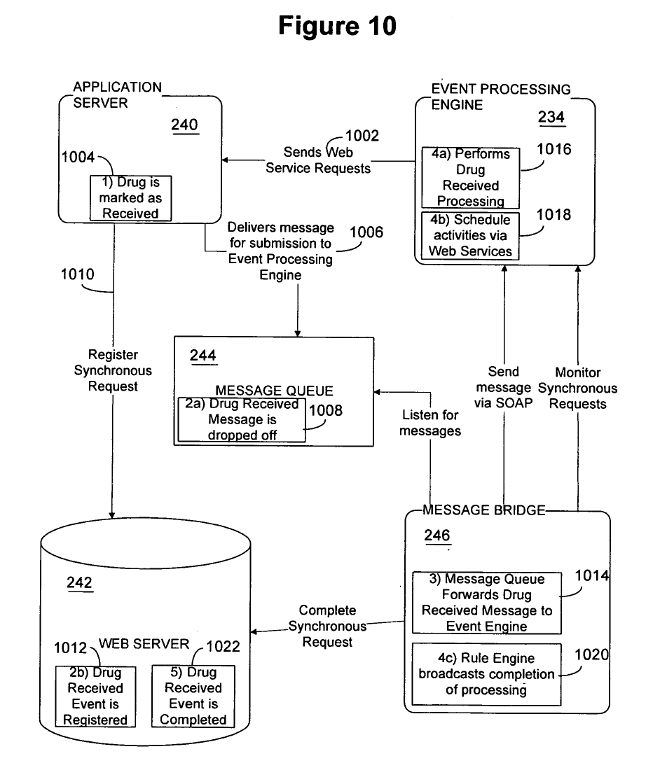

FIG. 10 is a functional diagram that depicts components executing in memory of the administrator system. These components are described in the context of the illustrative example of a patient scheduling a medical service. The components exhibit similar functionality in the scope of other types of services. In the illustrative example, the event processing engine component of the scheduler sends a request to the treatment location to determine whether a drug has been received (step 1002). The computer at the treatment location respond with a message indicating that the drug has been received (step 1004). This return message is received via the web site and captured by the application server. The application server places the received message in the message queue for delivery to the event processing engine (step 1006). The received message waits in the queue until it is scheduled forward to the event processing engine (step 1008). The application server also notifies the database server that the received message has arrived (step 1010). In turn, the database server registers receipt of the received message in the database (step 1012).

The message bridge listens for messages in the message queue and when it determines that the received message is in the message queue, forwards the received message to the event processing engine (step 1014). The received message may be transmitted to the event processing engine using, for example, the SOAP protocol. After the event processing engine receives the received message, the event processing engine processes the event (step 1016). In the illustrative example, the event processing engine identifies that the “drug ready” event has occurred and notifies parties to the medical treatment that the drug has been received (step 1018). The event processing engine registers completion of the “drug ready” event by sending a notification to the database server (step 1020) via the message bridge. Then, the database server records the completion of the event in the database (step 1022).

If the scheduler receives a message from a party that affects the timing of a scheduled appointment, then the scheduler modifies the appointment time and notifies the relevant parties. For example, if the scheduler receives a message from the treatment location that the drug has not been received (that is, the “drug ready” state has not occurred), then the scheduler notifies the provider that service is not ready and modifies the appointment time by a predetermined number of days in accordance with the event rules. For example, the schedule may move out the appointment by 30 or 60 days to allow the drug to arrive prior to the treatment. An illustrative event rule set for this case is shown in Table 6. When it is determined that the appointment must be rescheduled, the scheduler notifies the relevant parties, such as the patient, the provider, and the drug provider of the newly appointment time.

TABLE 6
Drug ‘Ready’ State

| | | Notify | Notify | |
|---|---|---|---|---|
| Parameter | Condition | (instructional) | (informational) | Action/Escalation |
| clinic location | missing ‘x’ | Clinical, | Admin | 1 - Site alert in RO (Clinical, |
| request 1 | requirements | information | | Admin) |
| | | technology | | 2 - IT: Scheduler allows appt. to be |
| | | | | scheduled but adds 30 days to |
| | | | | appointment date |
| | | | | 3 - Clinical admin contacts clinic |
| | | | | and tries to get clinic ‘ready’ |
| | | | | 4 - Once clinic ‘ready’, Admin |
| | | | | ‘approves’ clinic to ‘implemented’ |
| | | | | state |
| | | | | 5 - IT: verifies clinic available for |
| | | | | appt. scheduling via scheduler. |
| clinic location | missing ‘x’ | Clinical, | Admin | 1 - Site alert in RO (Clinical, |
| request 2 | requirements | information | | Admin) |
| | | technology | | 2 - IT: Scheduler allows appt. to be |
| | | | | scheduled but adds 60 days to |
| | | | | appt date |
| | | | | 3 - Clinical admin contacts clinic |
| | | | | and tries to get clinic ‘ready’ |
| | | | | 4 - Once clinic ‘ready’, Admin |
| | | | | ‘approves’ clinic to ‘implemented’ |
| | | | | state |
| | | | | 5 - IT: verifies clinic available for |
| | | | | appt. scheduling via scheduler. |

The above-described examples of event processing are illustrative examples. The event processing engine of the administrator program may perform similar processing for other types of events, such as events identified above.

Referring back to FIG. 9, after the service has been completed, the provider location notifies the scheduler (step 920). This is performed, for example, by a nurse at the provider submitting a completion notification via the administrator web site. The scheduler receives this notification and, in turn, notifies relevant parties that the service has been completed (step 922). For example, the scheduler notifies the payer and the patient's doctor and the service has been completed.

The foregoing description of an implementation of the invention has been presented for purposes of illustration and description. It is not exhaustive and does not limit the invention to the precise form disclosed. Modifications and variations are possible in light of the above teachings or may be acquired from practicing the invention. For example, the described implementation includes software but the present implementation may be implemented as a combination of hardware and software or hardware alone. The invention may be implemented with both object-oriented and non-object-oriented programming systems. The scope of the invention is defined by the claims and their equivalents.

What is claimed:

1. A method in a data processing system having a program for coordinating a service, the method comprising the steps of:
receiving a request to coordinate a service;
identifying at least one location of a plurality of locations at which the service can be performed and at least one time at which the at least one location is available to perform the service;
selecting a selected location from the identified at least one location to perform the service;
scheduling a time to perform the service at the selected location from the at least one time at which the selected location is available;
notifying a recipient of the service, the selected location, and at least one resource provider of the scheduled time;
after the scheduled time is scheduled and before the scheduled time, periodically determining whether the recipient of the service and the selected location are available for the service at the scheduled time;
after the scheduled time is scheduled and before the scheduled time, periodically determining whether a resource that is used during the service has arrived at the selected location before a predetermined time;
when it is determined that at least one of the recipient and the selected location are not available at the scheduled time, notifying the recipient, the selected location, and a provider of the resource that the scheduled time is rescheduled to an adjusted scheduled time; and
when it is determined that the resource has not arrived by the predetermined time, notifying the recipient, the selected location, and the provider of the resource that the scheduled time is rescheduled to the adjusted scheduled time.

2. The method of claim 1, further comprising the step of:
determining whether the recipient of the service is eligible to receive the service.

3. The method of claim 1, further comprising the step of:
notifying a payer of the service that the service is completed.

4. The method of claim 1, wherein the resource is a product used during the service.

5. The method of claim 1, wherein information on status of the recipient of the service, the selected location, and the resource are received asynchronously.

6. The method of claim 1, wherein the service is at least one of a medical service, a training service, a transportation service, a product delivery, a corrections service, an a consulting service.

7. A computer-readable medium containing instructions that cause a data processing system to perform a method for coordinating a service, the method comprising the steps of:
receiving a request to coordinate a service;
identifying at least one location of a plurality of locations at which the service can be performed and at least one time at which the at least one location is available to perform the service;
selecting a selected location from the identified at least one location to perform the service;
scheduling a time to perform the service at the selected location from the at least one time at which the selected location is available;
notifying a recipient of the service, the selected location, and at least one resource provider of the scheduled time;
after the scheduled time is scheduled and before the scheduled time, periodically determining whether the recipient of the service and the selected location are available for the service at the scheduled time;
after the scheduled time is scheduled and before the scheduled time, periodically determining whether a resource that is used during the service has arrived at the selected location before a predetermined time;
when it is determined that at least one of the recipient and the selected location are not available at the scheduled time, notifying the recipient, the selected location, and a provider of the resource that the scheduled time is rescheduled to an adjusted scheduled time; and
when it is determined that the resource has not arrived by the predetermined time, notifying the recipient, the selected location, and the provider of the resource that the scheduled time is rescheduled to the adjusted scheduled time.

8. The computer-readable medium of claim 7, further comprising the step of:
determining whether the recipient of the service is eligible to receive the service.

9. The computer-readable medium of claim 7, further comprising the step of:
notifying a payer of the service that the service is completed.

10. The computer-readable medium of claim 7, wherein the resource is a product used during the service.

11. The computer-readable medium of claim 7, wherein information on status of the recipient of the service, the selected location, and the resource are received asynchronously.

12. The computer-readable medium of claim 7, wherein the medical service is at least one of a medical service, a training service, a transportation service, a product delivery, a corrections service, an a consulting service.

13. A data processing system comprising:
a memory having a program for coordinating a service that
receives a request to coordinate a service,
identifies at least one location of a plurality of locations at which the service can be performed and at least one time at which the at least one location is available to perform the service,
selects a selected location from the identified at least one location to perform the service,
schedules a time to perform the service at the selected location from the at least one time at which the selected location is available,
notifies a recipient of the service, the selected location, and at least one resource provider of the scheduled time,
after the scheduled time is scheduled and before the scheduled time, periodically determines whether the recipient of the service and the selected location are available for the service at the scheduled time,
after the scheduled time is scheduled and before the scheduled time, periodically determines whether a resource that is used during the service has arrived at the selected location before a predetermined time,
when it is determined that at least one of the recipient and the selected location are not available at the scheduled time, notifies the recipient, the selected location, and a provider of the resource that the scheduled time is rescheduled to an adjusted scheduled time, and
when it is determined that the resource has not arrived by the predetermined time, notifies the recipient, the selected location, and the provider of the resource that the scheduled time is rescheduled to the adjusted scheduled time; and
a processing unit that runs the program.

14. The data processing system of claim 13, wherein the program determines whether the recipient of the service is eligible to receive the service.

15. The data processing system of claim 13, wherein the program notifies a payer of the service that the service is completed.

16. The data processing system of claim 13, wherein the resource is a product used during the service.

17. The data processing system of claim 13, wherein information on status of the recipient of the service, the selected location, and the resource are received asynchronously.

18. The data processing system of claim 13, wherein the medical service is at least one of a medical service, a training service, a transportation service, a product delivery, a corrections service, an a consulting service.
